(19) United States
(12) Patent Application Publication
(10) Pub. No.: US 2026/0083501 A1
(43) Pub. Date: Mar. 26, 2026

(54) TRANSCATHETER LASER DEVICE

(71) Applicant: Konstantinos SPARGIAS, Athens (GR)

(72) Inventor: Konstantinos SPARGIAS, Athens (GR)

(21) Appl. No.: 19/113,524

(22) PCT Filed: Sep. 20, 2023
(86) PCT No.: PCT/GR2023/000049

§ 371 Date: Mar. 20, 2025

(30) Foreign Application Priority Data
Sep. 22, 2022 (GR) 20220100774

(51) Int. Cl. A61B 18/24; A61B 18/00

(52) U.S. Cl. CPC A61B 18/24; A61B 2018/00369; A61B 2018/00577; A61B 2018/00601

(57) ABSTRACT

A catheter is configured to be inserted transvascularly into a body of a subject and advanced toward a leaflet of a native heart valve of the subject or a prosthetic heart valve implanted in the body of the subject. The catheter includes a distal segment including a distal tip, the distal segment configured to deflect so as to position the distal tip with respect to the leaflet. A pre-shaped nitinol wire guide loop is configured to be deployed and brought into contact with the leaflet. One or more optical fibers deliver laser energy to the leaflet via the distal tip when (i) the distal tip is positioned with respect to the leaflet and (ii) a laser coupled to a proximal end of the catheter is activated, and (iii) the proximal end of the catheter is disposed outside the body of the subject. Other embodiments are also described.

### CROSS-REFERENCE TO RELATED APPLICATIONS

The present application claims the priority of Greek Patent Application No. 20220100774 to Spargias, entitled, “A laser catheter device for transcatheter splitting, drilling and/or annihilation of a prosthetic or native heart valve leaflet,” filed Sep. 22, 2022, which is incorporated herein by reference.

### BACKGROUND

Transcatheter prosthetic valve replacement (or implantation) has been increasingly used as the preferred mode of treatment in several cardiac valve diseases. Large scale clinical trials have proven the value and the benefits of transcatheter aortic valve replacement (TAVR) and the current American and European Professional Societal Guidelines have this therapy well positioned within the treatment protocols. Transcatheter pulmonary valve replacement has also been utilized for a long time even prior to TAVR, and multiple transcatheter mitral and tricuspid valve designs are under development and testing with several ongoing clinical trials.

TAVR has also become a standard method of treatment of old degenerated surgical prosthetic valves and gradually, as they start to emerge, of degenerated TAVR devices. Coronary obstruction is a life-threatening complication that occurs in up to 3% of patients undergoing transcatheter aortic valve replacement (TAVR) for degenerated surgical bioprostheses (valve-in-valve—ViV—procedure). The occurrence of this complication in TAVR in TAVR procedures is estimated to be much higher, between 2%-68%, depending on the type of the first TAVR device. Supra-annular TAVR devices that are implanted in at least half of all procedures performed globally today have a higher risk in the range quoted. In a ViV or in TAVR in TAVR procedure the leaflets of the prior valve are displaced towards the coronary ostia, creating a closed cage potentially resulting in an obstruction of coronary blood flow, either directly blocking the blood flow into the coronary ostia or the flow into the corresponding sinus of Valsalva. It is expected that the occurrence of this complication will further increase in the future with the expansion of the need for TAVR in TAVR procedures.

A proposed and tested intervention to prevent this complication is to split the leaflet, which will move and obstruct the coronary flow just before the implantation of the new valve. However, the technique described to achieve it (BASILICA) is very demanding and cumbersome, requiring non-specific hardware and materials and considerable expertise. The development of purpose-made devices to easily achieve this splitting will offer a big advantage and will make the difference between success and failure in the treatment of these patients.

A similar device would also offer a solution to the well-known and characterized catastrophic complication of Left Ventricular Outflow Tract (LVOT) obstruction, which can often occur when a transcatheter prosthetic valve is implanted either in a native or in a prosthetic mitral valve. The mechanism of this is that the displaced anterior mitral valve leaflet may obstruct the LVOT when the anatomy allows for it. A proposed and tested intervention to prevent this complication is to split this leaflet just before the implantation of the new valve. However, the technique described to achieve it (LAMPOON) is very demanding and cumbersome, requiring nonspecific hardware and materials and considerable expertise. The development of purpose-made device to easily achieve this splitting will offer a big advantage and will make the difference between success and failure in the treatment of these patients.

### FIELD OF THE INVENTION

The abstract of the invention describes the clinical problems that require and await a solution. Namely, displacement of an aortic or mitral valve leaflet by a transcatheter prosthesis that will obstruct coronary flow or the Left Ventricular Outflow tract (LVOT) respectively.

This complication although rare, can still occur in patients undergoing Transcatheter Aortic Valve Replacement (TAVR) for native aortic valve stenosis (up to 1% or more in some cases). The native leaflet is displaced by the TAVR device to directly obstruct blood flow in a coronary artery. It becomes more frequent in Valve in surgical Valve (ViV) procedures with reports of up to 3% incidence even after appropriate screening for this. Of course, patients screened as high or certain risk are already excluded from this therapy and are not represented in this percentage.

However, we have just started recognizing the full scale of this problem a couple of years ago when the first patients with degenerated TAVR devices came back for treatment with TAVR in TAVR. Coronary obstruction appears to be much more of an issue here with estimations that up to 68% in some valve types will be in high coronary obstruction risk.

The reason is that most of the current TAVR devices are supra-annular and have leaflets that reach high in the aortic root and/or have high sealing skirts, and therefore when the second TAVR device is implanted, a new higher cage is formed within the aortic root reaching to or above the sinotubular junction. The risk of coronary obstruction then is not only directly by the leaflet but also indirectly from this cage obstructing blood flow into a coronary sinus from above. FIGS. 1A-B illustrate the estimated occurrence of this complication when performing TAVR in TAVR, subject to the type of the initially implanted device. As shown the estimated incidence will be more than 50% of cases of many types of TAVR devices that are often used today (more than half of the global market). If a solution to the problem is not found soon, there is a serious risk that patients with degenerated TAVR device will have no other option but to undergo open heart surgery. In addition, if there is no transcatheter option down the road when their first TAVR device degenerates, the expansion of TAVR to younger patients will be questioned. It is noted that FIGS. 1A-B are prior art. FIG. 1A is from J Am Coll Cardiol Intv 2020;13:2617-27, and FIG. 1B is from EuroIntervention 2020;16:e1005-e1013. (FIG. 1A shows computed tomography-identified risk of coronary obstruction due to sinus sequestration in redo TAVR. For the data shown in FIG. 1B, p is less than 0.001.)

Multiple reports have demonstrated the feasibility of bioprosthetic aortic valve leaflet laceration and splitting utilizing an electrified coronary wire to reduce the risk of coronary obstruction (BASILICA technique). However, this technique is cumbersome, technically challenging and is not widely utilized. Most recently, a dedicated transcatheter device for leaflet splitting with applying the same principle of mechanical splitting has been developed and tested (ShortCut, Pi-Cardia). This is a bulky 16F device with many metallic parts and a needle for controlled laceration and splitting in a single line of an aortic leaflet before the new TAVR device is inserted into the body. It cannot be positioned and used preliminarily or inserted with the TAVR device in place, nor after the implantation of the TAVR device.

Another area that splitting of a valve leaflet may be necessary is when implanting a transcatheter mitral valve. The anterior mitral valve leaflet may sometimes be displaced to obstruct the LVOT, which is a serious life-threatening complication. Multiple reports have demonstrated the feasibility of transcatheter anterior mitral valve leaflet laceration and splitting utilizing an electrified coronary wire to reduce the risk (LAMPOON technique). However, and similar to BASILICA, this technique is cumbersome and technically challenging and is not widely utilized.

### SUMMARY OF EMBODIMENTS

In accordance with some applications of the present invention, a laser catheter is inserted transvascularly and advanced toward a heart valve of a subject. The heart valve may be a native heart valve of the subject or a prosthetic heart valve that has been implanted in the body of the subject. When a new prosthetic valve is being implanted, a leaflet of the pre-existing valve (whether the native valve of the subject or a prior implanted prosthetic valve) may be displaced in such a manner as to obstruct coronary flow (in the case of the aortic valve) or flow through the Left Ventricular Outflow Tract (in the case of the mitral valve). In order to prevent these obstructions of blood-flow, the laser catheter utilizes laser energy to sever, split, drill through, cut, and/or ablate at least part of the leaflet of the pre-existing valve prior to the placement of the new prosthetic valve.

Thus, in accordance with some applications of the present invention, the laser catheter includes one or more optical fibers whose fiber tips deliver laser energy through a distal tip of the catheter, at a distal-most end of a distal segment. The distal segment of the catheter can typically be deflected in a controlled manner so as to place the tip of the catheter into contact with a leaflet of the heart valve. Once the tip of the catheter is positioned in contact with the leaflet, a laser at the proximal end of the catheter (outside the subject's body) is activated so that the one or more optical fibers deliver the laser energy directly to the leaflet resulting in a cutting, severing, splitting, drilling, and/or ablation of at least part of the leaflet.

There is therefore provided, in accordance with some applications of the present invention, an apparatus including:
a catheter configured to be inserted transvascularly into a body of a subject and advanced toward a leaflet of a heart valve selected from the group consisting of: a native heart valve of the subject, and a prosthetic heart valve implanted in the body of the subject, the catheter including:
   a distal segment comprising a distal tip, the distal segment configured to deflect so as to bring the distal tip into contact with the leaflet; and
   one or more optical fibers disposed within the catheter and configured to deliver laser energy to the leaflet via the distal tip when (i) the distal tip is in contact with the leaflet and (ii) a laser coupled to a proximal end of the catheter is activated, the proximal end of the catheter disposed outside the body of the subject.

For some applications, the apparatus further includes a capturing element actuator configured to be disposed external to the body of the subject, the distal tip including a capturing element including two arms configured to be actuated so as to capture the leaflet between the two arms by the capturing element actuator, and the one or more optical fibers are configured to deliver the laser energy to the leaflet via one or both of the arms of the capturing element.

For some applications, the capturing element actuator is configured to actuate the capturing element to achieve open and closed positions of the arms, and the capturing element is configured to capture the leaflet when the arms are in the closed position.

For some applications, the apparatus further includes a catheter actuator configured to be disposed external to the body of the subject, the distal segment of the catheter configured to telescopically deflect so as to bring the distal tip into contact with the leaflet, and the telescopic deflection is actuated by a catheter actuator.

For some applications, the distal segment that is configured to telescopically deflect includes a plurality of telescoping segments and the distal tip comprises a distalmost one of the telescoping segments.

For some applications, the catheter is configured to be advanced over a wire in order to reach the heart valve.

For some applications, the catheter is configured to convey the laser energy such that the laser energy severs at least part of the leaflet when the distal tip is in contact with the leaflet and the laser is activated.

For some applications, the catheter is configured to convey the laser energy such that the laser energy splits at least part of the leaflet when the distal tip is in contact with the leaflet and the laser is activated.

For some applications, the catheter is configured to convey the laser energy such that the laser energy drills through at least part of the leaflet when the distal tip is in contact with the leaflet and the laser is activated.

For some applications, the catheter is configured to convey the laser energy such that the laser energy cuts at least part of the leaflet when the distal tip is in contact with the leaflet and the laser is activated.

For some applications, the catheter is configured to convey the laser energy such that the laser energy ablates at least part of the leaflet when the distal tip is in contact with the leaflet and the laser is activated.

For some applications, the heart valve is (i) a native aortic valve of the subject, or (ii) a prosthetic aortic valve implanted in the body of the subject.

For some applications, the heart valve is (i) a native mitral valve of the subject, or (ii) a prosthetic mitral valve implanted in the body of the subject.

For some applications, the apparatus is for use with a leaflet to which an implant is attached, and the catheter is configured to convey the laser energy such that the laser energy cuts the implant off the leaflet when the distal tip is in contact with the leaflet and the laser is activated.

For some applications, the implant is a mitral valve clip.

The present invention will be more fully understood from the following detailed description of embodiments thereof, taken together with the drawings, in which:

### BRIEF DESCRIPTION OF THE DRAWINGS

FIGS. 1A-B are prior art graphs showing statistical information;

FIG. 2 shows some prior art examples of laser fibers;

FIG. 3 shows experimental results, in accordance with some applications of the present invention;

FIGS. 4A-E are schematic illustrations of a laser catheter that has a deflectable distal segment and a leaflet capturing element at a distal tip of the catheter, in accordance with some applications of the present invention;

FIGS. 5A-B are schematic illustrations of the laser catheter of FIGS. 4A-E with a guide wire to assist insertion and advancing of the laser catheter, in accordance with some applications of the present invention;

FIGS. 6A-E are schematic illustrations of a laser catheter that has a deflectable distal segment and a distal tip without a capturing element, in accordance with some applications of the present invention;

FIGS. 7A-B are schematic illustrations of the laser catheter of FIGS. 6A-E with a guide wire to assist insertion and advancing of the laser catheter, in accordance with some applications of the present invention;

FIGS. 8A-C are schematic illustration of the laser catheter of FIGS. 4A-E with the shape of the capturing element being a circle, in accordance with some applications of the present invention;

FIGS. 9A-E show the addition of a pre-shaped nitinol wire guide loop that exits the laser catheter and is deployed to conform deep into the sinus of Valsalva to guide and improve the position of the laser catheter in the sinus, in accordance with some applications of the present invention;

FIGS. 10A-C are schematic illustrations of a laser catheter for use in severing, splitting, drilling, and/or ablation of the anterior mitral valve leaflet, in accordance with some applications of the present invention;

FIG. 11 is a schematic illustration depicting multiple examples of layouts of laser fiber tips with the distal tip of the laser catheter, in accordance with some applications of the present invention; and

FIG. 12 is a schematic illustration of the mitral valve with a clip implant attached to its leaflets and the laser catheter for use in cutting the clip implant off one or both of the leaflets, in accordance with some applications of the present invention.

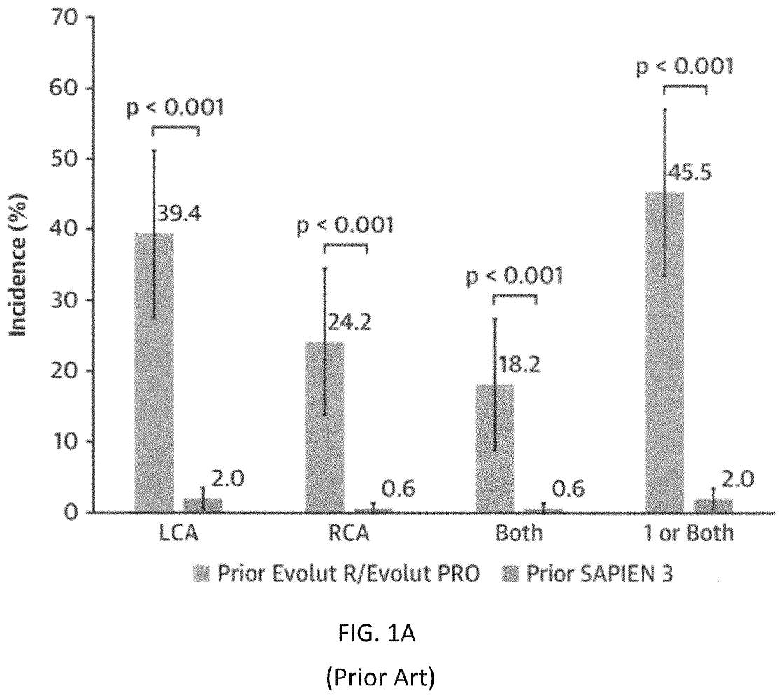

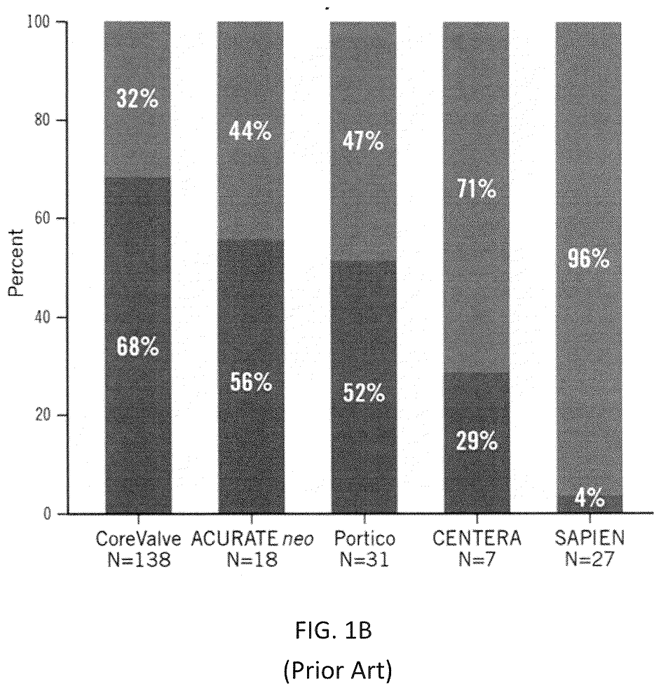

### DESCRIPTION OF THE INVENTION

Embodiments of the invention described aim to provide a dedicated transcatheter solution for easy and safe splitting and/or drilling a heart valve leaflet or ablating part of it using fiber delivered laser energy.

Laser energy has been safely used extensively for transmyocardial revascularization in the past decades, both via endocardial and epicardial approach. The concept of clinically beneficial laser revascularization has not been proved, but there has been extensive basic research on the effect of the various laser systems (XeCI excimer, dye, YAG, YAG pulsed, CO2 pulsed) and characteristics (wavelength, pulse domain, max pulse energy, max rep rate, max power, penetration depth, penetration diameter) on penetrating and channeling the myocardial tissue. In addition, it has been long used for recanalization of blocked or heavily obstructed coronary and peripheral arteries with an excellent safety record.

### Transcatheter Aortic Laser Leaflet Splitting (TALLS)

In accordance with some applications of the present invention, using the available data from previous medical experience, an arrangement of laser fibers can be used to deliver via the proposed laser leaflet splitter (LLS) the necessary energy to split, drill or ablate the leaflet tissue that would otherwise obstruct the coronary flow after the new TAVR device implantation. The aim here is to split the prosthetic aortic heart valve leaflet (either surgical or transcatheter) that is estimated to obstruct blood flow into the coronaries before implanting the new device.

The characteristics of the laser beam are typically carefully selected after testing so that the damage (ablation) to the tissue could reach up to about 1-5 mm distance from the tip(s) of one or more fibers and be completed within a few seconds.

The device uses laser energy conveyed to the intended location by one or multiple fibers within a purpose made catheter. The distal segment of this catheter may be deflectable to allow approaching and contacting the intended locations with ease.

The end of the catheter may have a capturing element in the general shape of the Greek letter lambda (Λ), that has the property of externally controlled closure or collapse of its sides to hold the leaflet from its edge to the desired length towards its base. In one (or both) inner face(s) of the capturing element there is disposed an array of straight or lateral emitting laser fibers.

Depending on the shape of the capturing element and the arrangement of the laser delivering fibers, it is possible to split the leaflet, drill a hole of any shape in it or ablate part of it. This is done when the external laser attached to the outer end of the catheter (i.e., the proximal end of the catheter that is external to the subject's body) is activated, with the capturing element still on the held leaflet (or slowly moving away, allowing the leaflet to slip gradually out while it is split).

Another embodiment may not have the capturing element at the catheter tip, rather the embodiment provides using the built-in deflection properties of the catheter to have the catheter approach and come into contact with the outflow (i.e., downstream) surface of the leaflet only and deliver the laser energy via an array of one or multiple laser fibers ending and emitting at its tip to achieve the same results. In this arrangement, the laser fiber straight or lateral emitting tips are aimed at the leaflet-facing surface of the last few millimeters of the catheter tip. The deflection helps the catheter to approach the upper and outer (downstream) surface of the targeted aortic leaflet, and then the release of the deflection brings the catheter tip (final segment) in contact with the upper (downstream) surface of the leaflet.

The array of the laser fibers in the above-described embodiments can be in the shape of a straight line and its activation would result in a straight line cutting and splitting of the attached leaflet. Other arrays of the fibers could be in the shape of a circle and its activation would result in drilling a hole in the leaflet. If the catheter is slowly withdrawn with the laser actuated, the hole would be extended towards the edge of the leaflet and could even result in a wide split.

All the described device embodiments could be advanced over a wire that would cross the aortic valve (or stay above it), or on their own using the built-in catheter deflection property.

This device could be also used to split a native aortic valve leaflet when it is estimated that it will obstruct coronary flow if displaced by a TAVR device.

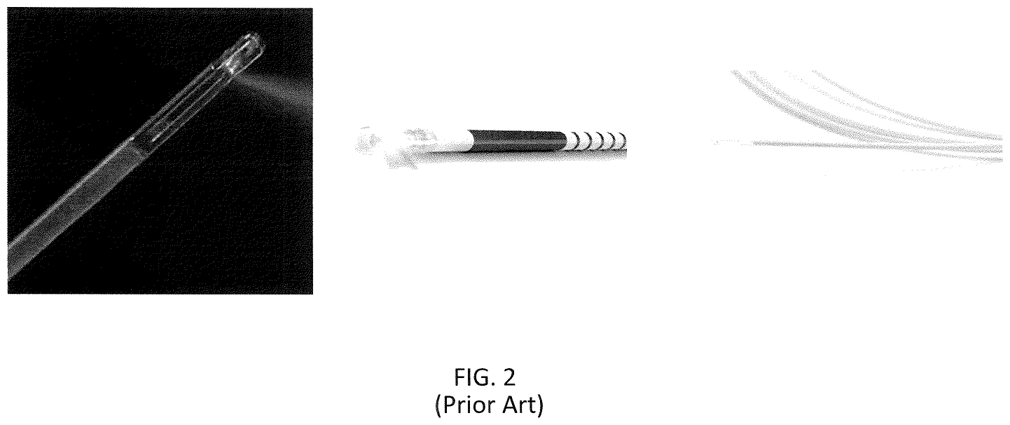

The laser fibers provided herein can be straight or lateral emitting (examples of some prior art laser fibers are shown in FIG. 2)

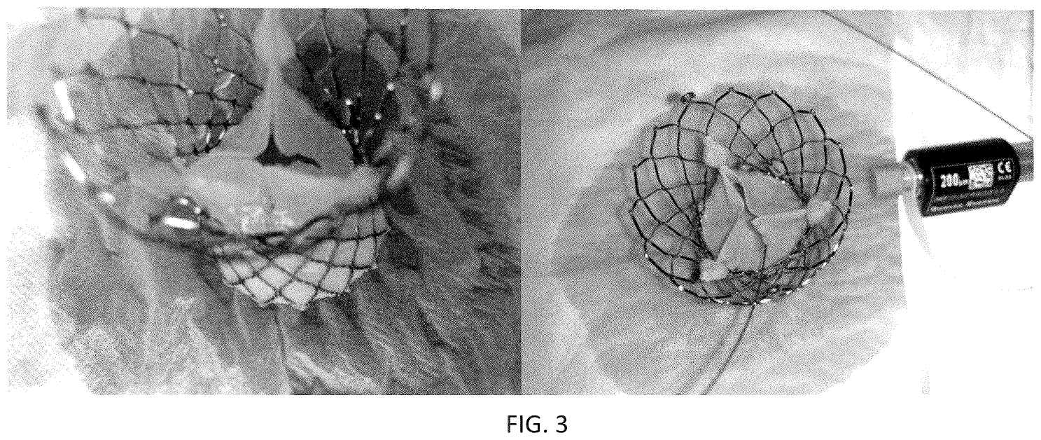

FIG. 3 shows results of an in-vitro experiment of a successful leaflet split of a CoreValve 34mm TAVR device (Medtronic, USA) with the use of a single Urology Laser Fiber 200 micron connected to an external laser providing a beam at 8 Hz and 1.5 mJ. It took only a few seconds to manually split the leaflet with this laser fiber, even though it is the thinnest and weakest of all urology fibers.

The capability of a single 200 micron core diameter quartz fiber to in vitro resect calcified aortic heart valve leaflets has already been shown by Rohde et al (J Card Surg 2015;30:157-162). A pulse energy of 4.3 mJ, a pulse duration of 0.8-1 microsecond and a repetition rate of 1 kHz was used and achieved a resection rate of 40.4 +/−22 mg/min, which corresponds to a cutting speed of 1 cm/min. The majority of the remnant particles (85.4%) were <6 microns, and only 0.1% exceeded 300 microns. These results suggest that the in vivo application should be safe with regards to particle embolization complications.

Past animal experiments in vivo have shown that the entire excision of the aortic valve leaflets with laser fibers is feasible by both transfemoral and transapical approach and without noticeable adjacent tissue damage (Quaden et al. Interact CardioVasc Thorac Surg 2012;15:348-351 and Zhang Sai, https://macau.uni-kiel.de/receive/diss_mods_00003604?lang=en).

The outer diameter of the commonly used laser fibers in urology are 1.2F-2F (for 200-900 micron fibers). Therefore, the described applications of the present invention utilizing similar fibers could for example incorporate 1 to 6 fibers and provide a catheter diameter of 2F-12F, which could make it very easy to use. An array of a few fibers in a row activated simultaneously would shorten the time required to split a leaflet to a minimum. In addition, this may reduce or even negate the need to move/slide the device in order to achieve ablation of adjoining tissue. The fibers in a row can be straight or lateral emitting.

### Transcatheter Mitral Laser Leaflet Splitting (TMLLS)

A similar concept to the above-described LLS for use in aortic valve leaflets, is used for splitting of the native anterior mitral valve leaflet when it is anticipated to obstruct the LVOT when displaced by a transcatheter mitral valve prosthesis.

A deflectable LLS catheter is inserted over a wire into the LVOT. The array of the emitting laser fiber tips is now aimed at the leaflet-facing surface of the deflectable tip of the catheter. Under fluoroscopic and transesophageal guidance, this surface of the catheter tip is brought to full contact with the distal part of the anterior mitral valve leaflet (its LVOT surface) Then laser is activated and the anterior leaflet is split. The transcatheter mitral valve can then be implanted safely with the risk of LVOT obstruction ameliorated.

Another embodiment of this device provides an approach to the mitral valve from the left atrium through atrial transseptal puncture and access. It provides inherent steerable capabilities to direct its tip to the leaflet area that needs to be split.

Finally, a similar device can be used to cut an attached implant off one or more mitral or tricuspid valve leaflets and fully or partially release it from them (for example a MitraClip or PASCAL clip). This is useful after a procedure of transcatheter leaflet edge-to-edge repair with such devices fails to reduce valve regurgitation and reintervention is required but the patient cannot undergo conventional surgery. In such a case, as provided by these applications of the present invention, the described transcatheter laser device approaches the valve from its atrial or ventricular surface, comes in touch with it, and delivers energy from laser fibers at its tip area to cut the leaflet close and around the implant and release the implant from the leaflet. Then the implant attached to the other leaflet can be left in place, and a transcatheter valve can be implanted. Alternatively, the implant can be cut off both leaflets and removed from the body using a special capture mechanism and a sheath.

### Detailed Description of the Drawings

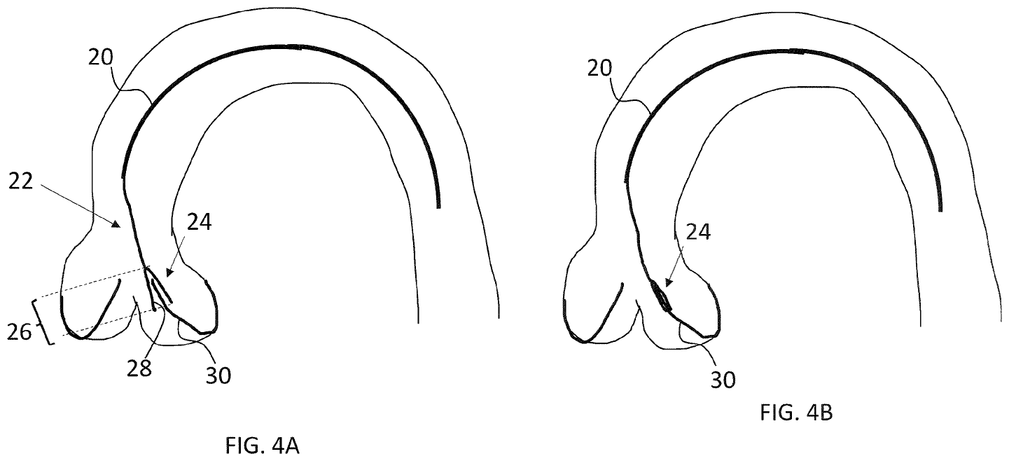

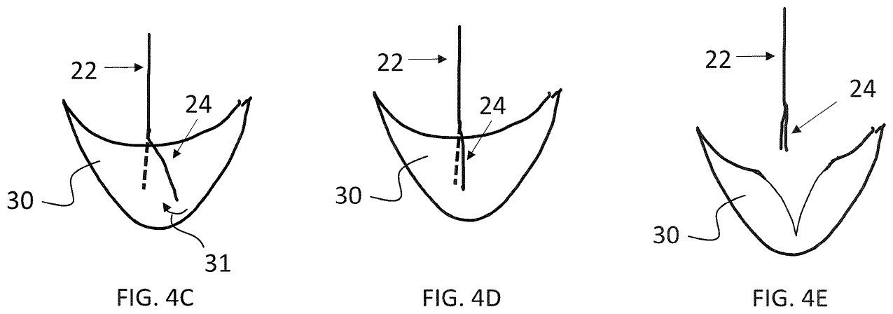

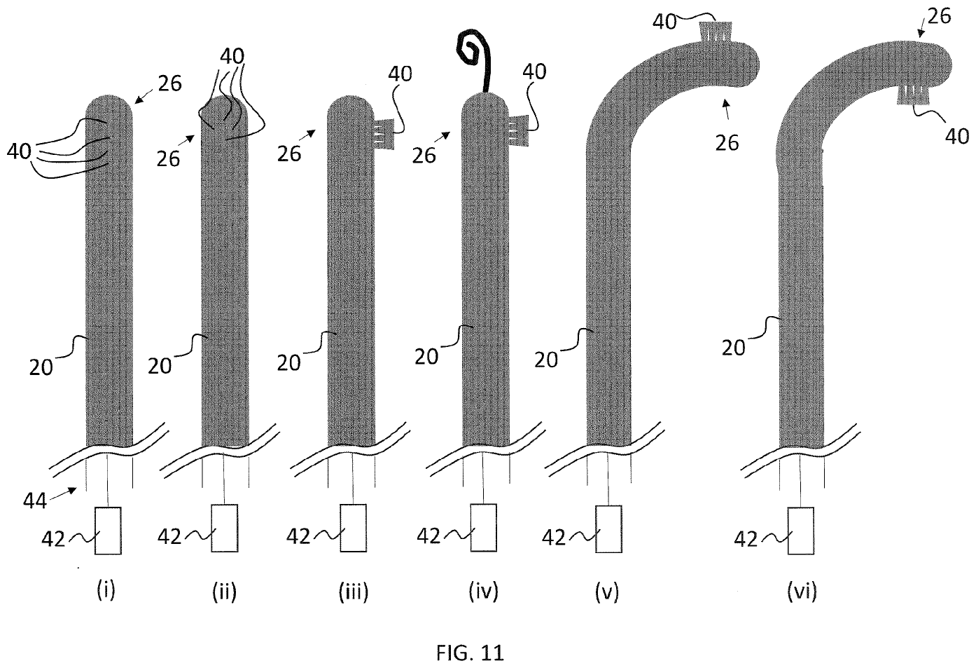

Reference is now made to FIGS. 4A-E, which depict a laser catheter 20 that has a deflectable distal segment 22 and a leaflet capturing element 24 (opening and closing) at a distal tip 26 of catheter 20. One or multiple straight or lateral emitting optical fibers 40 (also referred to herein as laser fibers) travelling within catheter 20 reach the inner surface 28 of capturing element 24 (in one or both of its arms). The leaflet 30 is captured, typically under fluoroscopy and transesophageal echocardiography guidance. Then the laser energy is applied by activating a laser 42 (shown in FIG. 11) and leaflet 30 is split. Catheter 20 may stay still or be withdrawn slowly during this process to ensure complete split of leaflet 30 to its edge. The laser activation time required for successful splitting depends on the laser fiber and source characteristics and the energy delivered, and is typically a few seconds. FIG. 4A shows capturing element 24 in an open position with its arms on either side of leaflet 30. FIG. 4B shows capturing element 24 in a closed position in which it has captured leaflet 30 between its arms. FIG. 4C shows a front view of leaflet 30 and capturing element 24 in an open position, with one arm on each side of leaflet 30. FIG. 4D shows the same front view of leaflet 30, with capturing element 24 now in a closed position. Arrow 31 in FIG. 4C shows the direction of motion of an arm of capturing element 24 as it moves from the open position shown in FIG. 4C to the closed position shown in FIG. 4D. FIG. 4E shows the same front view of leaflet 30 after the laser has been activated and leaflet 30 cut.

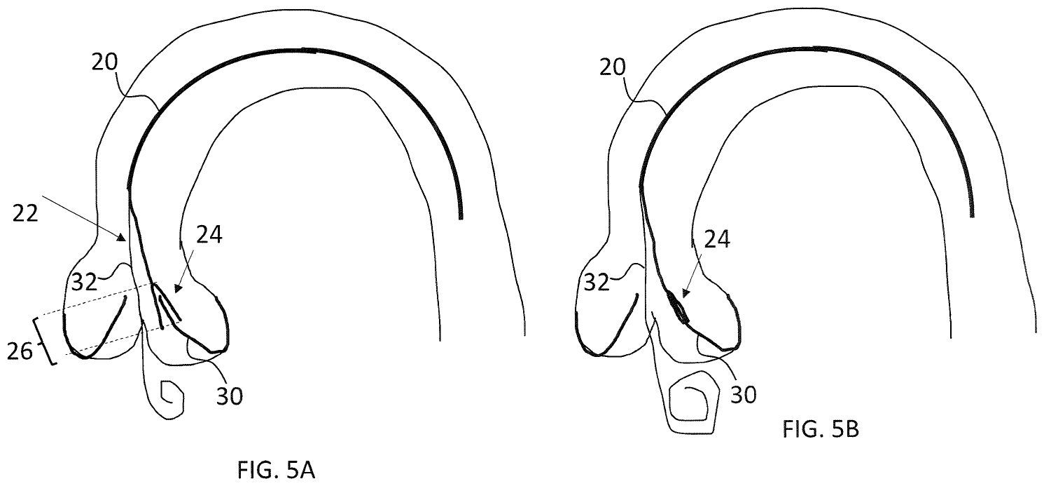

Reference is now made to FIGS. 5A-B, which depict what is described earlier with reference to FIGS. 4A-E but with the addition of a guide wire 32 to assist insertion and advancing of the laser catheter 20 to the intended location. FIG. 5A shows capturing element 24 in an open position with its arms on either side of leaflet 30. FIG. 5B shows capturing element 24 in a closed position in which it has captured leaflet 30 between its arms.

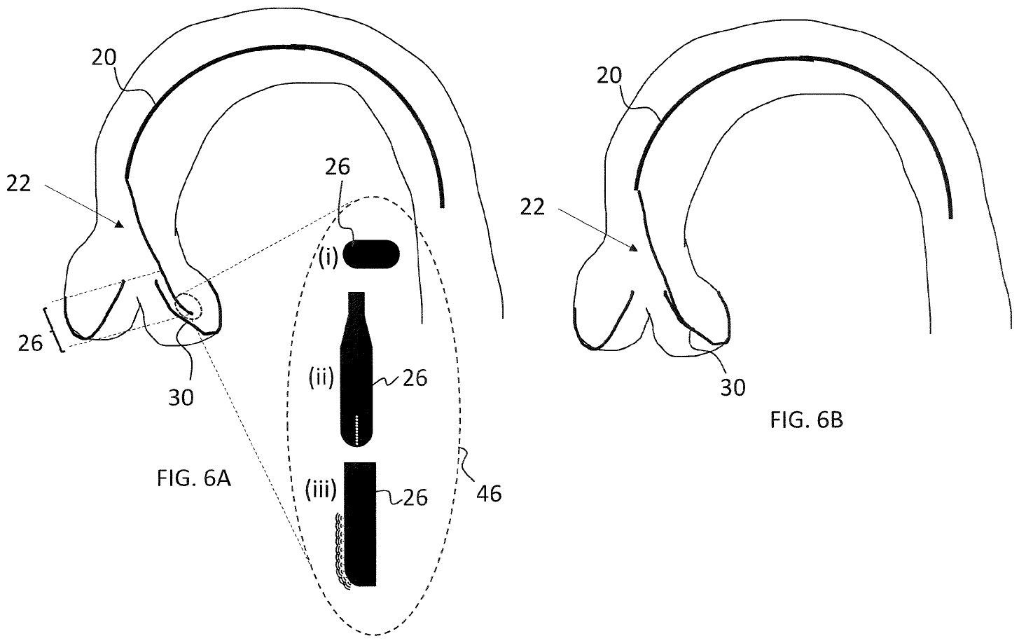

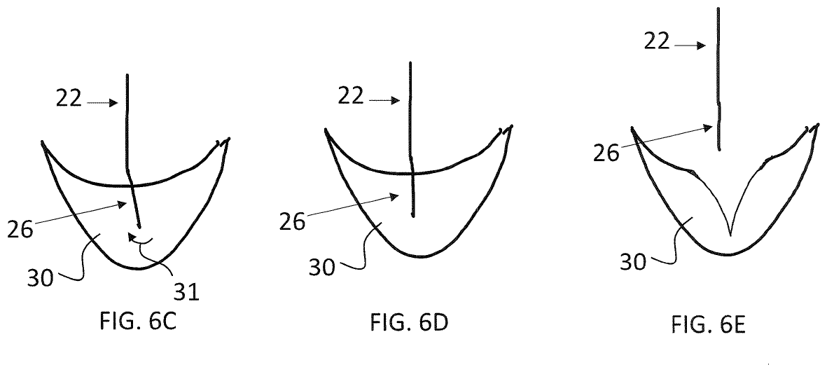

Reference is now made to FIGS. 6A-E, which depict laser catheter 20 without leaflet capturing element 24 at distal tip 26 of catheter 20. Here the laser fibers array is at the leaflet-facing surface of distal tip 26. Catheter 20 is deflected and guided above the aortic surface of leaflet 30, then the deflection is gradually released until it comes in complete contact with a sufficient length of leaflet 30. The laser is then activated, and leaflet 30 (in contact with distal tip 26) is split. FIG. 6A shows distal tip 26 not in contact with leaflet 30. FIG. 6B shows distal tip 26 in contact with leaflet 30. FIG. 6C shows a front view of leaflet 30 and distal tip 26 not in contact with leaflet 30. FIG. 6D shows the same front view of leaflet 30, with distal tip 26 now in contact with leaflet 30. Arrow 31 in FIG. 6C shows the direction of motion of distal tip 26 as the deflection is gradually released and distal tip 26 moves from the position shown in FIG. 6C to the position shown in FIG. 6D. FIG. 6E shows the same front view of leaflet 30 after the laser has been activated and leaflet 30 cut. Enlarged view 46 in FIG. 6A shows (i) a cross-section of distal tip 26, (ii) a coronal section of distal tip 26, and (iii) a sagittal section of distal tip 26.

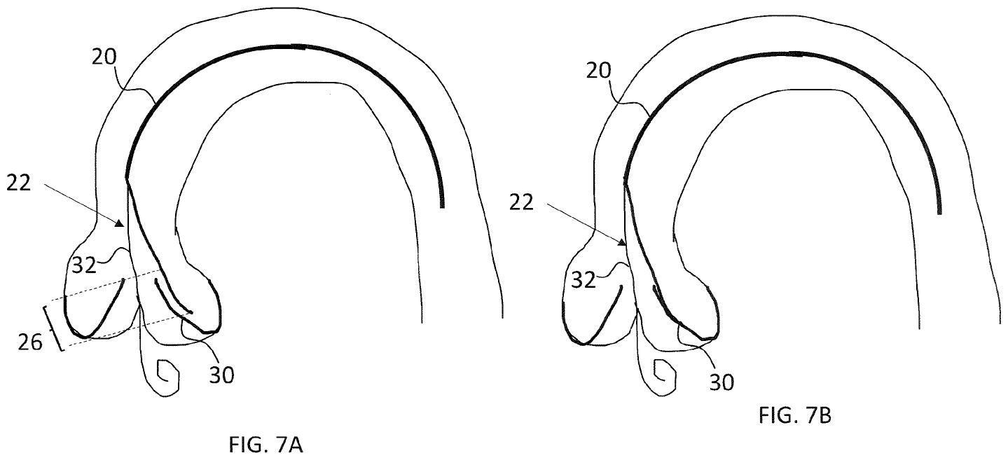

Reference is now made to FIGS. 7A-B, which depict what is described in FIGS. 6A-B but with the addition of guide wire 32 to assist insertion and advancing of laser catheter 20 to the intended location. FIG. 7A shows distal tip 26 not in contact with leaflet 30. FIG. 7B shows distal tip 26 in contact with leaflet 30.

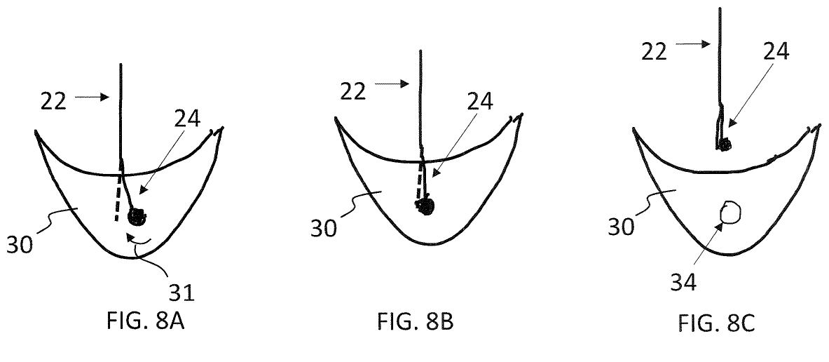

Reference is now made to FIGS. 8A-C, which depict what is described earlier with reference to FIGS. 4A-B but with the shape of capturing element 24 at distal tip 26 being a circle that corresponds to the position of the laser fibers. Therefore, when the laser is activated it drills a hole 34 of that size in leaflet 30. The catheter may stay still or be withdrawn slowly during this process to cause the leaflet drilling to cause a wide split of leaflet 30. FIG. 8A shows a front view of leaflet 30 and capturing element 24 in an open position, with one arm on each side of leaflet 30. FIG. 8B shows the same front view of leaflet 30 with capturing element 24 now in a closed position. Arrow 31 in FIG. 8A shows the direction of motion of an arm of capturing element 24 as it moves from the open position shown in FIG. 8A to the closed position shown in FIG. 8B. FIG. 8C shows the same front view of leaflet 30 after the laser has been activated and hole 34 drilled in leaflet 30.

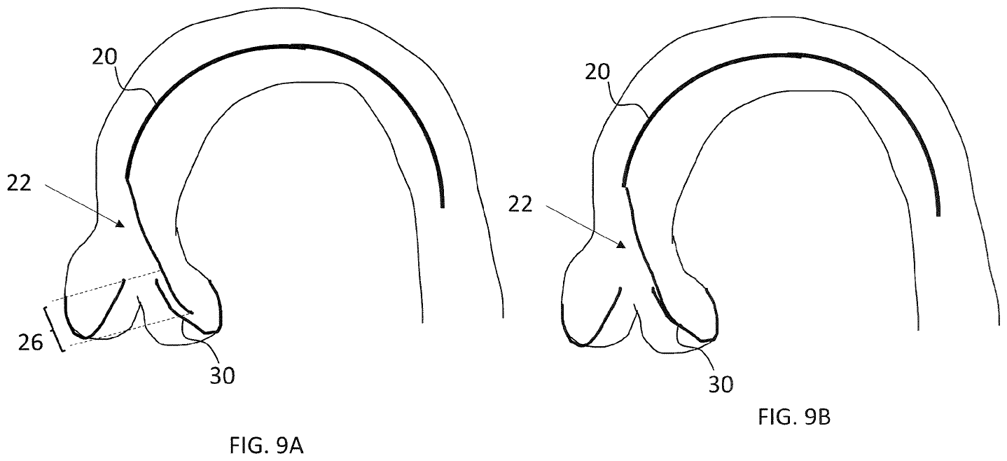

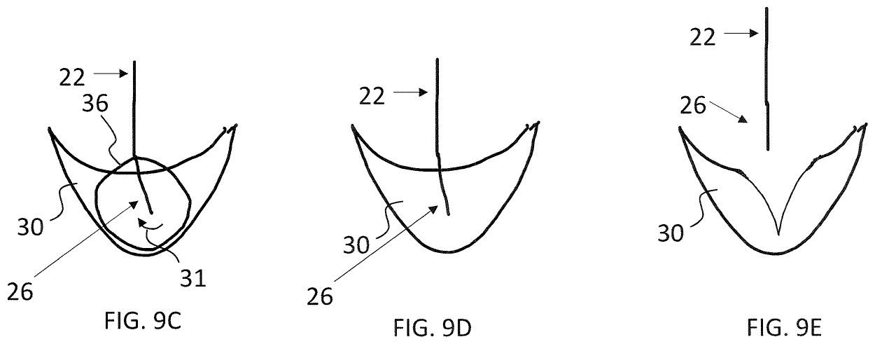

Reference is now made to FIGS. 9A-E, which depict the addition of a pre-shaped nitinol wire guide loop 36 that exits catheter 20 and is deployed to conform deep into the sinus of Valsalva to guide and improve the position of the laser catheter 20 in the sinus. Then laser catheter 20 is advanced to reach and contact the intended location on the aortic surface of leaflet 30. The pre-shaped nitinol wire guide loop 36 may then be withdrawn back into the catheter 20. In FIG. 9A, pre-shaped nitinol wire guide loop 36 is deployed and positioned generally in the same plane as distal tip 26 (and is therefore not easily seen, in contrast to the view of FIG. 9C). Distal tip 26 is not in contact with leaflet 30. FIG. 9B shows distal tip 26 in contact with leaflet 30, with pre-shaped nitinol wire guide loop 36 having been withdrawn back into catheter 20. FIG. 9C shows a front view of leaflet 30 and expanded pre-shaped nitinol wire guide loop 36 with distal tip 26 not in contact with leaflet 30. FIG. 9D shows the same front view of leaflet 30 with distal tip 26 in contact with leaflet 30 with pre-shaped nitinol wire guide loop 36 having been withdrawn back into catheter 20. Arrow 31 in FIG. 9C shows the direction of motion of distal tip 26 as the deflection is gradually released and distal tip 26 moves from the position shown in FIG. 9C to the position shown in FIG. 9D. FIG. 9E shows the same front view of leaflet 30 after the laser has been activated and leaflet 30 cut. It is noted that although FIGS. 9A-E depict distal tip 26 of catheter 20 without capturing element 24, pre-shaped nitinol wire guide loop 36 may be utilized with any of the embodiments described herein for catheter 20.

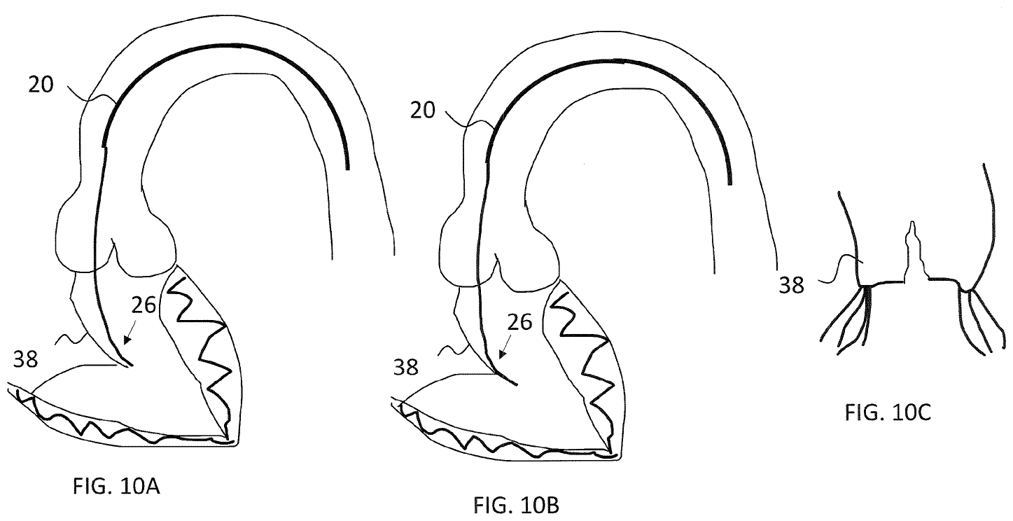

Reference is now made to FIGS. 10A-C, which depict a catheter 20 for use in severing, splitting, drilling, and/or ablation of the anterior mitral valve leaflet 38. Catheter 20 is advanced over a wire into the LVOT, as shown in FIG. 10A, and then deflected to approach and come into contact with the LVOT surface of anterior mitral valve leaflet 38 (from its tip towards its base), as shown in FIG. 10B. Then laser energy is delivered by the laser fiber array aimed at the leaflet-facing surface of catheter tip 26. FIG. 10C shows a front view of anterior mitral valve leaflet 38 after the laser has been activated and leaflet 38 cut.

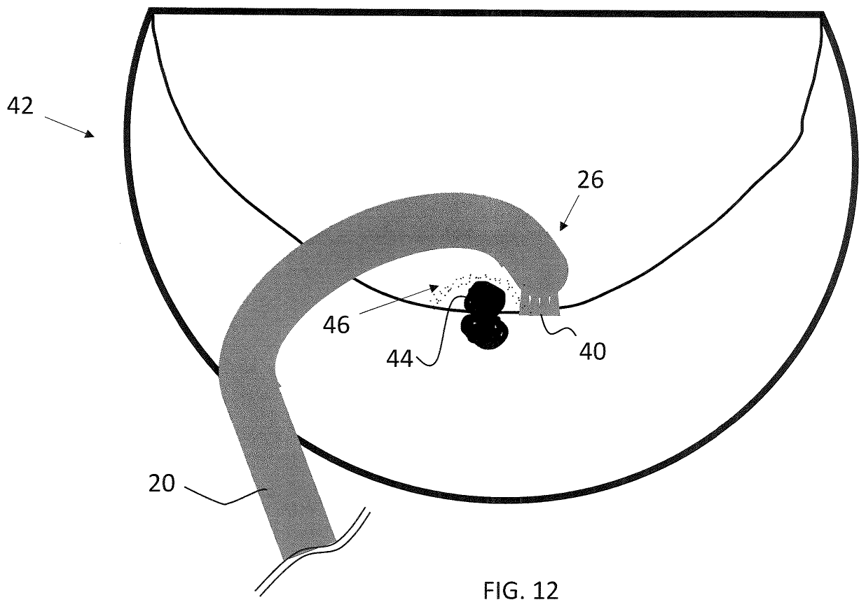

Reference is now made to FIG. 11, which depicts multiple examples of potential layouts of the tips of optical fibers 40 at distal tip 26 of catheter 20, in accordance with some applications of the present invention. The fiber tips can be in linear, such as is shown in example (i), circular, such as is shown in example (ii), or any other pattern. The laser emitting surface can be opposite the deflecting direction, i.e., on the outer curved surface of distal tip 26 such as is shown in example (v) (possibly most suitable use for TALLS), or at the deflecting direction, i.e., on the inner curved surface of distal tip 26 such as is shown in example (vi) (possibly most suitable use for TMLLS). All catheters can be either self-advanced or advanced over a wire. Laser 42 is coupled to a proximal end 44 of catheter 20. Proximal end 44 of catheter 20 is disposed outside the body of the subject. Reference is now made to FIG. 12, which depicts the mitral valve 42 (atrial or surgeons view) with a clip device 44 attached to its leaflets in the center. Distal tip 26 of laser catheter 20 approaches via transseptal puncture, and deflects and steers to touch and deliver laser energy along the border line to the clip body at the anterior leaflet, cutting off the clip from one or both of the leaflets. The path of the laser energy is depicted by series of dots 46.

### Inventive Concepts

1. A catheter that comprises one or multiple traversing laser fibers, is inserted transvascularly and is advanced to reach a native or prosthetic heart valve and bring to contact its fibers'emitting tips with a surface of a valve leaflet in order to split/drill/ablate the leaflet with the emitted laser energy provided by a source mounted on the external end of the catheter.
2. A catheter as described in inventive concept 1, that has a capturing element at its tip, which can be externally piloted to attain open and closed position and contains the array of the laser fiber emitting tips in the inner surface of one or both arms. The targeted leaflet is actively or passively captured into this element and the leaflet tissue within is split/drilled/ablated with laser energy.
3. A catheter as described in inventive concepts 1 and 2, that has the property of externally driven telescopic deflection of its distal segment so that it can be guided to the intended location to facilitate attaching/affixing and/or capturing to the targeted leaflet.
4. A catheter as described in inventive concepts 1-3, that has the property of externally driven telescopic deflection of its distal segment so that it can be guided to the intended location to come into contact with the targeted leaflet surface. The array of the laser fiber emitting tips are at the inner surface (opposite of the deflecting direction) of its very final tip (last few millimeters).
5. A catheter as described in inventive concepts 1-4, that has the array of the laser fiber emitting tips at the outer surface (direction of the deflection) of its very final tip (last few millimeters).
6. A catheter as described in inventive concepts 1-5, that is advanced over a wire to reach the intended location close to the targeted valve leaflet. This wire can be placed within the left ventricle or stay out of it above the aortic valve.
7. A catheter as described in inventive concepts 1-6, that will be used for severing a native or prosthetic aortic valve leaflet.
8. A catheter as described in inventive concepts 1-7, that will be used for severing a native or prosthetic mitral valve leaflet.
9. A catheter as described in inventive concepts 1-8, that will be used for severing a native or prosthetic heart valve leaflet.
10. A catheter as described in inventive concepts 1-9, that will be used for cutting off a device attached to one or more valve leaflets. If this device is completely detached it will then be captured by a lasso wire or other manner and removed from the body through a larger sheath that accommodates the catheter.

It will be appreciated by persons skilled in the art that the present invention is not limited to what has been particularly shown and described hereinabove. Rather, the scope of the present invention includes both combinations and subcombinations of the various features described hereinabove, as well as variations and modifications thereof that are not in the prior art, which would occur to persons skilled in the art upon reading the foregoing description.

1-15. (canceled)

16. An apparatus comprising:
a catheter configured to be inserted transvascularly into a body of a subject and advanced toward a leaflet of a heart valve selected from the group consisting of: a native heart valve of the subject, and a prosthetic heart valve implanted in the body of the subject, the catheter comprising:
a distal segment comprising a distal tip, the distal segment configured to deflect so as to position the distal tip with respect to the leaflet;
a pre-shaped nitinol wire guide loop configured to be deployed and brought into contact with the leaflet; and
one or more optical fibers disposed within the catheter and configured to deliver laser energy to the leaflet via the distal tip when (i) the distal tip is positioned with respect to the leaflet, (ii) a laser coupled to a proximal end of the catheter is activated, and (iii) the proximal end of the catheter is disposed outside the body of the subject.

17. The apparatus according to claim 16, wherein the apparatus further comprises a wire and the catheter is configured to be advanced over the wire in order to reach the heart valve.

18. The apparatus according to claim 16, wherein the catheter is configured to convey the laser energy such that the laser energy severs at least part of the leaflet when the distal tip is positioned with respect to the leaflet and the laser is activated.

19. The apparatus according to claim 16, wherein the catheter is configured to convey the laser energy such that the laser energy splits at least part of the leaflet when the distal tip is positioned with respect to the leaflet and the laser is activated.

20. The apparatus according to claim 16, wherein the catheter is configured to convey the laser energy such that the laser energy drills through at least part of the leaflet when the distal tip is positioned with respect to the leaflet and the laser is activated.

21. The apparatus according to claim 16, wherein the catheter is configured to convey the laser energy such that the laser energy cuts at least part of the leaflet when the distal tip is positioned with respect to the leaflet and the laser is activated.

22. The apparatus according to claim 16, wherein the catheter is configured to convey the laser energy such that the laser energy ablates at least part of the leaflet when the distal tip is positioned with respect to the leaflet and the laser is activated.

23. The apparatus according to claim 16, wherein the heart valve is (i) a native aortic valve of the subject, or (ii) a prosthetic aortic valve implanted in the body of the subject, and wherein the catheter is configured to be advanced toward the native aortic valve or the prosthetic aortic valve.

24. The apparatus according to claim 16, wherein the heart valve is (i) a native mitral valve of the subject, or (ii) a prosthetic mitral valve implanted in the body of the subject, and wherein the catheter is configured to be advanced toward the native mitral valve or the prosthetic mitral valve.

25. The apparatus according to claim 16, wherein the leaflet is a leaflet to which an implant is attached, and the catheter is configured to convey the laser energy such that the laser energy cuts the implant off the leaflet to which the implant is attached, when the distal tip is positioned with respect to the leaflet and the laser is activated.

26. The apparatus according to claim 25, wherein the implant is a mitral valve clip
